(19) United States
(12) Patent Application Publication
(10) Pub. No.: US 2020/0056226 A1
(43) Pub. Date: Feb. 20, 2020

(54) METHOD FOR DIAGNOSING PARKINSON'S DISEASE THROUGH BACTERIAL METAGENOME ANALYSIS

(71) Applicant: MD HEALTHCARE INC., Seoul (KR)

(72) Inventor: Yoon-Keun KIM, Gyeonggi-do (KR)

(21) Appl. No.: 16/488,263

(22) PCT Filed: Feb. 23, 2018
(86) PCT No.: PCT/KR2018/002281

§ 371 Date: Aug. 23, 2019

(30) Foreign Application Priority Data
Feb. 24, 2017 (KR) 10-2017-0025001
Feb. 22, 2018 (KR) 10-2018-0021196

(51) Int. Cl. C12Q 1/689

(52) U.S. Cl. CPC C12Q 1/689

(57) ABSTRACT

The present invention relates to a method of diagnosing Parkinson's disease through bacterial metagenomic analysis, and more particularly, to a method of diagnosing Parkinson's disease by analyzing an increase or decrease in content of extracellular vesicles derived from specific bacteria by bacterial metagenomic analysis using a subject-derived sample. Extracellular vesicles secreted from microorganisms such as bacteria, archaea, and the like present in the environment are absorbed into the human body and distributed in the brain, and thus may directly affect inflammatory responses and brain functions, and since it is difficult to implement early diagnosis for Parkinson's disease, which is characterized by inflammation, before symptoms appear, efficient treatment therefor is difficult. Thus, according to the present invention, a risk for developing Parkinson's disease may be predicted through metagenomic analysis of bacteria-derived extracellular vesicles using a human body-derived sample, and thus the onset of Parkinson's disease may be delayed or prevented through appropriate management by early diagnosis and prediction of a risk group for Parkinson's disease, and, even after Parkinson's disease occurs, early diagnosis for Parkinson's disease may be implemented, thereby lowering the incidence rate of Parkinson's disease and increasing therapeutic effects.

### TECHNICAL FIELD

The present invention relates to a method of diagnosing Parkinson's disease through bacterial metagenomic analysis, and more particularly, to a method of diagnosing Parkinson's disease by analyzing an increase or decrease in content of extracellular vesicles derived from specific bacteria by bacterial metagenomic analysis using a subject-derived sample.

### BACKGROUND ART

Parkinson's disease is a progressive neurodegenerative disease characterized by Parkinsonian symptoms such as slow motion, tremors at rest, muscle stiffness, abnormal gait characteristics, and a bending posture. The disease occurs due to a decrease in stimulation of the motor cortex which results from imperfect dopamine production and action, mainly in the substantia nigra. Serious cognitive impairment and mild language impairment also occur and they are chronic and progressive. Parkinson's disease may also occur even when contracting Japanese encephalitis, brain syphilis, carbon dioxide poisoning, manganese poisoning, or Wilson's disease. The probability of developing the disease is one in 1,000, but incidence increases with age. In addition, movement disorders occur, making it uncomfortable to move.

Unlike most other serious neurological or psychological disorders, Parkinson's disease has a relatively low hereditary nature. Most patients do not have relatives with Parkinson's disease. In a case in which one of the identical twins contracts the disease, the probability for the other person to contract the disease is not even 10%. This is, of course, clearly higher than a 0.1% probability that other people contract the disease, but is not so high. Incidence-related environmental factors include blockage of blood flowing to specific areas of the brain, long-term exposure to certain drugs and poisons, and history of being infected by encephalitis or other viruses.

Meanwhile, it is known that the number of microorganisms symbiotically living in the human body is 100 trillion which is 10 times the number of human cells, and the number of genes of microorganisms exceeds 100 times the number of human genes. A microbiota is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-0073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of Parkinson's disease, identification of causative factors of Parkinson's disease through metagenomic analysis of bacteria-derived vesicles isolated from a human-derived substance, such as urine and the like, and a method of diagnosing Parkinson's disease have never been reported.

### DISCLOSURE

### Technical Problem

To pre-diagnose causative factors of Parkinson's disease and a risk of developing the disease, the inventors of the present invention extracted DNA from bacteria-derived extracellular vesicles present in urine, which is a subject-derived sample, and performed metagenomic analysis on the extracted DNA, and, as a result, identified bacteria-derived extracellular vesicles capable of acting as a causative factor of Parkinson's disease, and thus completed the present invention based on these findings.

Therefore, an object of the present invention is to provide a method of providing information for Parkinson's disease diagnosis through metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

To achieve the above-described object of the present invention, there is provided a method of providing information for Parkinson's disease diagnosis, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject sample with that of a normal individual-derived sample through sequencing of a product of the PCR.

The present invention also provides a method of diagnosing Parkinson's disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject sample with that of a normal individual-derived sample through sequencing of a product of the PCR.

The present invention also provides a method of predicting a risk for Parkinson's disease, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject sample with that of a normal individual-derived sample through sequencing of a product of the PCR.

In one embodiment of the present invention, the subject sample may be urine.

In another embodiment of the present invention, process (c) may comprise comparing an increase or decrease in content of extracellular vesicles derived from one or more bacteria selected from the group consisting of the phylum Proteobacteria, the phylum Cyanobacteria, the phylum Gemmatimonadetes, the phylum Chloroflexi, the phylum Synergistetes, the phylum Acidobacteria, the phylum Planctomycetes, the phylum OD1, the phylum WS3, the phylum Parvarchaeota, the phylum OP1, the phylum Chlorobi, the phylum OP9, the phylum Hyd24-12, and the phylum Thermotogae.

In another embodiment of the present invention, process (c) may comprise comparing an increase or decrease in content of extracellular vesicles derived from one or more bacteria selected from the group consisting of the class Coriobacteriia, the class Gammaproteobacteria, the class Verrucomicrobiae, the class Actinobacteria, the class Alphaproteobacteria, the class Chloroplast, the class Saprospirae, the class Deltaproteobacteria, the class Epsilonproteobacteria, the class Ellin6529, the class Chloracidobacteria, the class Opitutae, the class Thermoleophilia, the class Synergistia, the class Gemmatimonadetes, the class Planctomycetia, the class Acidobacteriia, the class Solibacteres, the class Anaerolineae, the class Chloroflexi, the class Phycisphaerae, the class Synechococcophycideae, the class TM7-1, the class Acidimicrobiia, the class Acidobacteria-6, the class Spartobacteria, the class ABY1, the class Pedosphaerae, the class ZB2, the class PRR-12, the class Ktedonobacteria, the class JS1, the class WM88, the class Dehalococcoidetes, the class SAR202, and the class MSBL6.

In another embodiment of the present invention, process (c) may comprise comparing an increase or decrease in content of extracellular vesicles derived from one or more bacteria selected from the group consisting of the order RF39, the order Turicibacterales, the order Pseudomonadales, the order Coriobacteriales, the order Pasteurellales, the order Enterobacteriales, the order Verrucomicrobiales, the order Gemellales, the order Neisseriales, the order Saprospirales, the order Actinomycetales, the order Streptophyta, the order Rhizobiales, the order Rhodospirillales, the order Xanthomonadales, the order Myxococcales, the order Campylobacterales, the order Desulfovibrionales, the order Solirubrobacterales, the order Opitutales, the order RB41, the order Rickettsiales, the order Pirellulales, the order Synergistales, the order Planctomycetales, the order Acidobacteriales, the order Solibacterales, the order Gaiellales, the order Gemmatales, the order Acidimicrobiales, the order WD2101, the order Chthoniobacterales, the order Thermoanaerobacterales, the order Pedosphaerales, the order Phycisphaerales, the order Sediment-1, the order Chlorophyta, the order iii1-15, the order Synechococcales, the order Roseiflexales, the order JG30-KF-AS9, the order Ellin329, the order Anaerolineales, the order Ellin5290, the order SC-I-84, the order Cryptophyta, the order MBNT15, the order envOPS12, the order B07_WMSP1, the order UA01, and the order Thermotogales.

In another embodiment of the present invention, process (c) may comprise comparing an increase or decrease in content of extracellular vesicles derived from one or more bacteria selected from the group consisting of the family Exiguobacteraceae, the family Enterococcaceae, the family Turicibacteraceae, the family Mogibacteriaceae, the family Moraxellaceae, the family Porphyromonadaceae, the family Burkholderiaceae, the family Actinomycetaceae, the family Coriobacteriaceae, the family Methylobacteriaceae, the family Streptococcaceae, the family Pseudomonadaceae, the family Pasteurellaceae, the family Veillonellaceae, the family Peptostreptococcaceae, the family Enterobacteriaceae, the family Verrucomicrobiaceae, the family Lachnospiraceae, the family Leuconostocaceae, the family Bradyrhizobiaceae, the family Rikenellaceae, the family Tissierellaceae, the family Bacteroidaceae, the family Chitinophagaceae, the family Corynebacteriaceae, the family Xanthomonadaceae, the family Rhizobiaceae, the family Propionibacteriaceae, the family Desulfobacteraceae, the family Barnesiellaceae, the family Comamonadaceae, the family mitochondria, the family Hyphomicrobiaceae, the family Alteromonadaceae, the family Sinobacteraceae, the family Pirellulaceae, the family Dethiosulfovibrionaceae, the family Acidobacteriaceae, the family Planctomycetaceae, the family Isosphaeraceae, the family Gaiellaceae, the family Koribacteraceae, the family Helicobacteraceae, the family Chthoniobacteraceae, the family Gemmataceae, the family C111, the family Solibacteraceae, the family Pelagibacteraceae, the family PRR-10, the family Ellin515, the family Thermoanaerobacteraceae, the family Methanoregulaceae, the family Synechococcaceae, the family Desulfomicrobiaceae, the family Kouleothrixaceae, the family OCS155, the family Alicyclobacillaceae, the family Myxococcaceae, the family EB1017, the family Anaerolinaceae, and the family Desulfohalobiaceae.

In another embodiment of the present invention, process (c) may comprise comparing an increase or decrease in content of extracellular vesicles derived from one or more bacteria selected from the group consisting of the genus Collinsella, the genus Adlercreutzia, the genus SMB53, the genus Proteus, the genus Exiguobacterium, the genus Enterococcus, the genus Acinetobacter, the genus Turicibacter, the genus Klebsiella, the genus Lautropia, the genus Akkermansia, the genus Parabacteroides, the genus Rhizobium, the genus Actinomyces, the genus Lactococcus, the genus Blautia, the genus Veillonella, the genus Pseudomonas, the genus Rothia, the genus Dorea, the genus Streptococcus, the genus Haemophilus, the genus Enhydrobacter, the genus Rhodococcus, the genus Coprococcus, the genus Oscillospira, the genus Ruminococcus, the genus Bacteroides, the genus Corynebacterium, the genus Weissella, the genus Propionibacterium, the genus Lysinibacillus, the genus Stenotrophomonas, the genus Arthrobacter, the genus Comamonas, the genus Marinobacter, the genus Clostridium, the genus Planctomyces, the genus Luteolibacter, the genus Delftia, the genus Agrobacterium, the genus Rhodoplanes, the genus DA101, the genus Gemmata, the genus Coprobacillus, the genus Arcobacter, the genus Helicobacter, the genus Candidatus solibacter, the genus Methanosarcina, the genus Thermacetogenium, the genus Synechococcus, the genus Desulfomicrobium, the genus Chthoniobacter, the genus Aminobacterium, the genus Gallicola, the genus Anaeromyxobacter, the genus Muricauda, and the genus Candidatus Koribacter.

### Advantageous Effects

Extracellular vesicles secreted from microorganisms such as bacteria, archaea, and the like present in the environment are absorbed into the human body, and thus can directly affect the occurrence of inflammation, and since it is difficult to implement early diagnosis for Parkinson's disease, which is characterized by inflammatory responses, before symptoms appear, efficient treatment therefor is difficult. Thus, according to the present invention, a risk for developing Parkinson's disease may be predicted through metagenomic analysis of bacteria-derived extracellular vesicles using a human body-derived sample, and thus the onset of Parkinson's disease can be delayed or prevented through appropriate management by early diagnosis and prediction of a risk group for Parkinson's disease, and, even after Parkinson's disease occurs, early diagnosis for Parkinson's disease can be implemented, thereby lowering the incidence rate of Parkinson's disease and increasing therapeutic effects. In addition, patients diagnosed with Parkinson's disease are able to avoid exposure to causative factors predicted through metagenomic analysis, whereby the progression of Parkinson's disease is ameliorated, or recurrence thereof can be prevented.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, blood and various organs were extracted.

FIG. 2 illustrates distribution results of bacteria-derived EVs exhibiting significant diagnostic performance at a phylum level, after metagenomic analysis of bacteria-derived EVs isolated from Parkinson's disease patient-derived urine and normal individual-derived urine.

FIG. 3 illustrates distribution results of bacteria-derived EVs exhibiting significant diagnostic performance at a class level, after metagenomic analysis of bacteria-derived EVs isolated from Parkinson's disease patient-derived urine and normal individual-derived urine.

FIG. 4 illustrates distribution results of bacteria-derived EVs exhibiting significant diagnostic performance at an order level, after metagenomic analysis of bacteria-derived EVs isolated from Parkinson's disease patient-derived urine and normal individual-derived urine.

FIG. 5 illustrates distribution results of bacteria-derived EVs exhibiting significant diagnostic performance at a family level, after metagenomic analysis of bacteria-derived EVs isolated from Parkinson's disease patient-derived urine and normal individual-derived urine.

FIG. 6 illustrates distribution results of bacteria-derived EVs exhibiting significant diagnostic performance at a genus level, after metagenomic analysis of bacteria-derived EVs isolated from Parkinson's disease patient-derived urine and normal individual-derived urine.

### BEST MODE

The present invention relates to a method of diagnosing Parkinson's disease through bacterial metagenomic analysis. The inventors of the present invention extracted genes from bacteria-derived extracellular vesicles using a subject-derived sample, performed metagenomic analysis thereon, and identified bacteria-derived extracellular vesicles capable of acting as a causative factor of Parkinson's disease.

Thus, the present invention provides a method of providing information for Parkinson's disease diagnosis, the method comprising:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles of the subject sample with that of a normal individual-derived sample through sequencing of a product of the PCR.

The term “Parkinson's disease diagnosis” as used herein refers to determining whether a patient has a risk for Parkinson's disease, whether the risk for Parkinson's disease is relatively high, or whether Parkinson's disease has already occurred. The method of the present invention may be used to delay the onset of Parkinson's disease through special and appropriate care for a specific patient, which is a patient having a high risk for Parkinson's disease or prevent the onset of Parkinson's disease. In addition, the method may be clinically used to determine treatment by selecting the most appropriate treatment method through early diagnosis of Parkinson's disease.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

The term “bacteria-derived vesicles” as used herein is a concept comprising extracellular vesicles derived from bacteria or archaea, but the present invention is not limited thereto.

In the present invention, the subject sample may be urine, but the present invention is not limited thereto.

In an embodiment of the present invention, metagenomic analysis is performed on the bacteria-derived extracellular vesicles, and bacteria-derived extracellular vesicles capable of acting as a cause of the onset of Parkinson's disease were actually identified by analysis at phylum, class, order, family, and genus levels.

More particularly, in one embodiment of the present invention, as a result of performing bacterial metagenomic analysis on extracellular vesicles present in subject-derived urine samples at a phylum level, the content of extracellular vesicles derived from bacteria belonging to the phylum Proteobacteria, the phylum Cyanobacteria, the phylum Gemmatimonadetes, the phylum Chloroflexi, the phylum Synergistetes, the phylum Acidobacteria, the phylum Planctomycetes, the phylum OD1, the phylum WS3, the phylum Parvarchaeota, the phylum OP1, the phylum Chlorobi, the phylum OP9, the phylum Hyd24-12, and the phylum Thermotogae was significantly different between Parkinson's disease patients and normal individuals (see Example 4).

More particularly, in one embodiment of the present invention, as a result of performing bacterial metagenomic analysis on extracellular vesicles present in subject-derived urine samples at a class level, the content of extracellular vesicles derived from bacteria belonging to the class Coriobacteriia, the class Gammaproteobacteria, the class Verrucomicrobiae, the class Actinobacteria, the class Alphaproteobacteria, the class Chloroplast, the class Saprospirae, the class Deltaproteobacteria, the class Epsilonproteobacteria, the class Ellin6529, the class Chloracidobacteria, the class Opitutae, the class Thermoleophilia, the class Synergistia, the class Gemmatimonadetes, the class Planctomycetia, the class Acidobacteriia, the class Solibacteres, the class Anaerolineae, the class Chloroflexi, the class Phycisphaerae, the class Synechococcophycideae, the class TM7-1, the class Acidimicrobiia, the class Acidobacteria-6, the class Spartobacteria, the class ABY1, the class Pedosphaerae, the class ZB2, the class PRR-12, the class Ktedonobacteria, the class JS1, the class WM88, the class Dehalococcoidetes, the class SAR202, and the class MSBL6 was significantly different between Parkinson's disease patients and normal individuals (see Example 4).

More particularly, in one embodiment of the present invention, as a result of performing bacterial metagenomic analysis on extracellular vesicles present in subject-derived urine samples at an order level, the content of extracellular vesicles derived from bacteria belonging to the order RF39, the order Turicibacterales, the order Pseudomonadales, the order Coriobacteriales, the order Pasteurellales, the order Enterobacteriales, the order Verrucomicrobiales, the order Gemellales, the order Neisseriales, the order Saprospirales, the order Actinomycetales, the order Streptophyta, the order Rhizobiales, the order Rhodospirillales, the order Xanthomonadales, the order Myxococcales, the order Campylobacterales, the order Desulfovibrionales, the order Solirubrobacterales, the order Opitutales, the order RB41, the order Rickettsiales, the order Pirellulales, the order Synergistales, the order Planctomycetales, the order Acidobacteriales, the order Solibacterales, the order Gaiellales, the order Gemmatales, the order Acidimicrobiales, the order WD2101, the order Chthoniobacterales, the order Thermoanaerobacterales, the order Pedosphaerales, the order Phycisphaerales, the order Sediment-1, the order Chlorophyta, the order iii1-15, the order Synechococcales, the order Roseiflexales, the order JG30-KF-AS9, the order Ellin329, the order Anaerolineales, the order Ellin5290, the order SC-I-84, the order Cryptophyta, the order MBNT15, the order envOPS12, the order B07_WMSP1, the order UA01, and the order Thermotogales was significantly different between Parkinson's disease patients and normal individuals (see Example 4).

More particularly, in one embodiment of the present invention, as a result of performing bacterial metagenomic analysis on extracellular vesicles present in subject-derived urine samples at a family level, the content of extracellular vesicles derived from bacteria belonging to the family Exiguobacteraceae, the family Enterococcaceae, the family Turicibacteraceae, the family Mogibacteriaceae, the family Moraxellaceae, the family Porphyromonadaceae, the family Burkholderiaceae, the family Actinomycetaceae, the family Coriobacteriaceae, the family Methylobacteriaceae, the family Streptococcaceae, the family Pseudomonadaceae, the family Pasteurellaceae, the family Veillonellaceae, the family Peptostreptococcaceae, the family Enterobacteriaceae, the family Verrucomicrobiaceae, the family Lachnospiraceae, the family Leuconostocaceae, the family Bradyrhizobiaceae, the family Rikenellaceae, the family Tissierellaceae, the family Bacteroidaceae, the family Chitinophagaceae, the family Corynebacteriaceae, the family Xanthomonadaceae, the family Rhizobiaceae, the family Propionibacteriaceae, the family Desulfobacteraceae, the family Barnesiellaceae, the family Comamonadaceae, the family mitochondria, the family Hyphomicrobiaceae, the family Alteromonadaceae, the family Sinobacteraceae, the family Pirellulaceae, the family Dethiosulfovibrionaceae, the family Acidobacteriaceae, the family Planctomycetaceae, the family Isosphaeraceae, the family Gaiellaceae, the family Koribacteraceae, the family Helicobacteraceae, the family Chthoniobacteraceae, the family Gemmataceae, the family C111, the family Solibacteraceae, the family Pelagibacteraceae, the family PRR-10, the family Ellin515, the family Thermoanaerobacteraceae, the family Methanoregulaceae, the family Synechococcaceae, the family Desulfomicrobiaceae, the family Kouleothrixaceae, the family OCS155, the family Alicyclobacillaceae, the family Myxococcaceae, the family EB1017, the family Anaerolinaceae, and the family Desulfohalobiaceae was significantly different between Parkinson's disease patients and normal individuals (see Example 4).

More particularly, in one embodiment of the present invention, as a result of performing bacterial metagenomic analysis on extracellular vesicles present in subject-derived urine samples at a genus level, the content of extracellular vesicles derived from bacteria belonging to the genus Collinsella, the genus Adlercreutzia, the genus SMB53, the genus Proteus, the genus Exiguobacterium, the genus Enterococcus, the genus Acinetobacter, the genus Turicibacter, the genus Klebsiella, the genus Lautropia, the genus Akkermansia, the genus Parabacteroides, the genus Rhizobium, the genus Actinomyces, the genus Lactococcus, the genus Blautia, the genus Veillonella, the genus Pseudomonas, the genus Rothia, the genus Dorea, the genus Streptococcus, the genus Haemophilus, the genus Enhydrobacter, the genus Rhodococcus, the genus Coprococcus, the genus Oscillospira, the genus Ruminococcus, the genus Bacteroides, the genus Corynebacterium, the genus Weissella, the genus Propionibacterium, the genus Lysinibacillus, the genus Stenotrophomonas, the genus Arthrobacter, the genus Comamonas, the genus Marinobacter, the genus Clostridium, the genus Planctomyces, the genus Luteolibacter, the genus Delftia, the genus Agrobacterium, the genus Rhodoplanes, the genus DA101, the genus Gemmata, the genus Coprobacillus, the genus Arcobacter, the genus Helicobacter, the genus Candidatus Solibacter, the genus Methanosarcina, the genus Thermacetogenium, the genus Synechococcus, the genus Desulfomicrobium, the genus Chthoniobacter, the genus Aminobacterium, the genus Gallicola, the genus Anaeromyxobacter, the genus Muricauda, and the genus Candidatus Koribacter was significantly different between Parkinson's disease patients and normal individuals (see Example 4).

From the above-described example results, it was confirmed that the identified distribution variables of the bacterial-derived extracellular vesicles were able to be effectively used to predict the occurrence of Parkinson's disease.

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Intestinal Bacteria and Bacteria-Derived Extracellular Vesicles

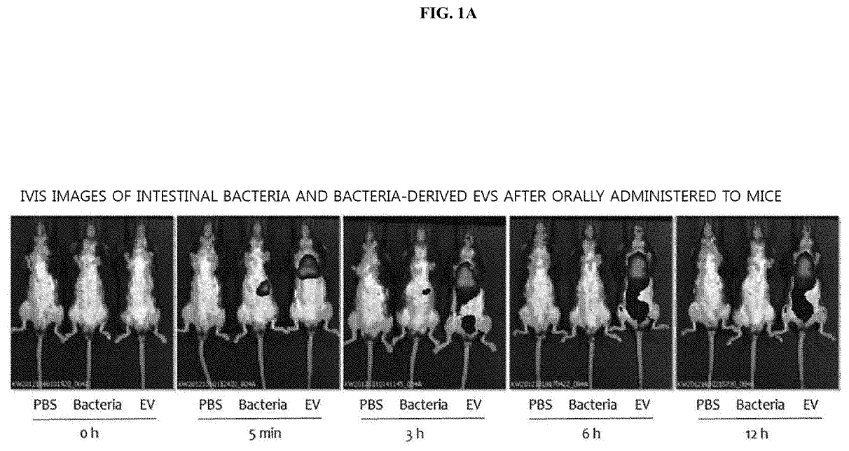

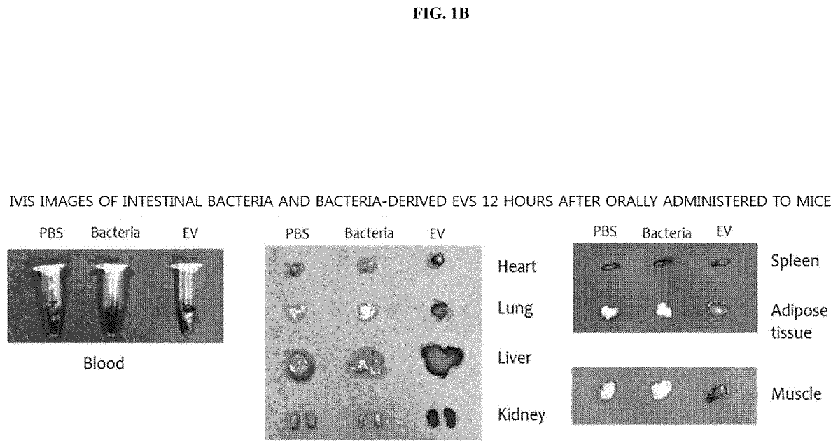

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction from Urine

To isolate extracellular vesicles and extract DNA, from urine, first, urine was added to a 10 ml tube and centrifuged at 3,500×g and 4 □ for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500×g and 4 □ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4 □ for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the urine according to the above-described method was boiled at 100 □ to allow the internal DNA to come out of the lipid and then cooled on ice. Next, the resulting vesicles were centrifuged at 10,000×g and 4 □ for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| | | | SEQ |
|---|---|---|---|
| Primer | | Sequence | ID NO. |
| 16S rDNA | 16S_V3_F | 5′-TCGTCGGCAGCGTCAGATGTG | 1 |
| | | TATAAGAGACAGCCTACGGGNGGC | |
| | | WGCAG-3′ | |
| | 16S_V4_R | 5′-GTCTCGTGGGCTCGGAGATGT | 2 |
| | | GTATAAGAGACAGGACTACHVGGG | |
| | | TATCTAATCC-3′ | |

### Example 3. Metagenomic Analysis Using DNA Extracted from Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score<20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Parkinson's Disease Diagnostic Model Based on Metagenomic Analysis of Bacteria-Derived EVs Isolated from Urine

EVs were isolated from urine samples of 39 Parkinson's disease patients and 76 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

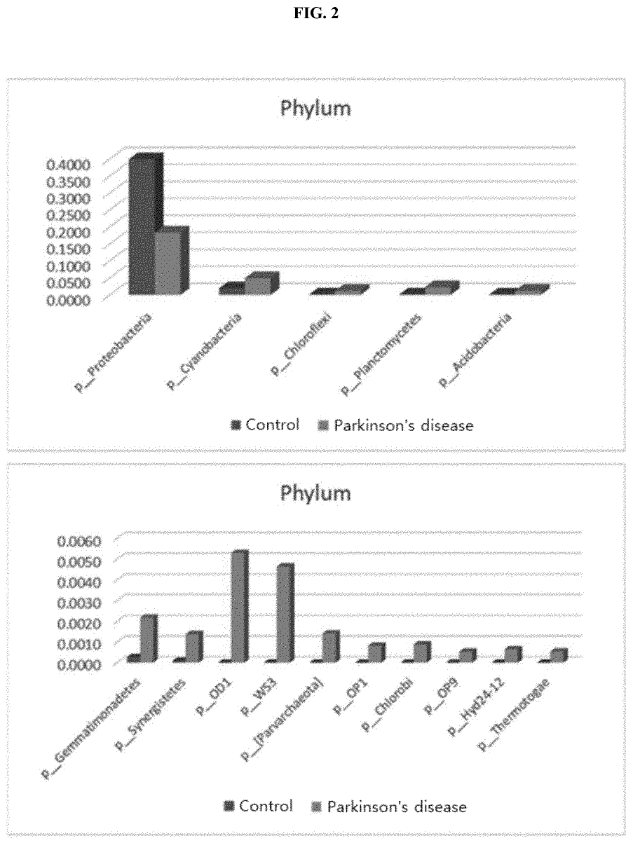

As a result of analyzing bacteria-derived EVs in urine at a phylum level, a diagnostic model developed using bacteria belonging to the phylum Proteobacteria, the phylum Cyanobacteria, the phylum Gemmatimonadetes, the phylum Chloroflexi, the phylum Synergistetes, the phylum Acidobacteria, the phylum Planctomycetes, the phylum OD1, the phylum WS3, the phylum Parvarchaeota, the phylum OP1, the phylum Chlorobi, the phylum OP9, the phylum Hyd24-12, and the phylum Thermotogae as a biomarker exhibited significant diagnostic performance for Parkinson's disease (see Table 2 and FIG. 2).

TABLE 2

| | | | Parkinson's | | | | | | |
|---|---|---|---|---|---|---|---|---|---|
| | Control | | disease | | t-test | | | | |
| Taxon | Mean | SD | Mean | SD | p-value | Ratio | AUC | Sensitivity | Specificity |
| p_Proteobacteria | 0.3993 | 0.1837 | 0.1836 | 0.0974 | 0.0000 | 0.46 | 0.95 | 0.86 | 0.92 |
| p_Cyanobacteria | 0.0191 | 0.0207 | 0.0486 | 0.0184 | 0.0000 | 2.55 | 0.87 | 0.86 | 0.54 |
| p_Gemmatimonadetes | 0.0003 | 0.0010 | 0.0022 | 0.0021 | 0.0000 | 8.03 | 0.81 | 0.93 | 0.64 |
| p_Chloroflexi | 0.0010 | 0.0030 | 0.0116 | 0.0052 | 0.0000 | 11.79 | 0.95 | 0.95 | 0.87 |
| p_Synergistetes | 0.0001 | 0.0003 | 0.0014 | 0.0018 | 0.0001 | 15.89 | 0.81 | 0.96 | 0.49 |
| p_Acidobacteria | 0.0005 | 0.0012 | 0.0119 | 0.0049 | 0.0000 | 25.85 | 1.00 | 0.97 | 0.95 |
| p_Planctomycetes | 0.0006 | 0.0019 | 0.0222 | 0.0094 | 0.0000 | 37.99 | 1.00 | 0.97 | 0.92 |
| p_OD1 | 0.0000 | 0.0000 | 0.0053 | 0.0043 | 0.0000 | 1219.90 | 0.97 | 0.99 | 0.85 |
| p_WS3 | 0.0000 | 0.0000 | 0.0046 | 0.0027 | 0.0000 | | 1.00 | 1.00 | 1.00 |
| p_[Parvarchaeota] | 0.0000 | 0.0000 | 0.0014 | 0.0025 | 0.0000 | | 0.95 | 1.00 | 0.85 |
| p_OP1 | 0.0000 | 0.0000 | 0.0008 | 0.0014 | 0.0000 | | 0.80 | 1.00 | 0.51 |
| p_Chlorobi | 0.0000 | 0.0000 | 0.0009 | 0.0012 | 0.0000 | | 0.79 | 1.00 | 0.54 |
| p_OP9 | 0.0000 | 0.0000 | 0.0005 | 0.0009 | 0.0000 | | 0.76 | 1.00 | 0.49 |
| p_Hyd24-12 | 0.0000 | 0.0000 | 0.0006 | 0.0012 | 0.0000 | | 0.75 | 1.00 | 0.44 |
| p_Thermotogae | 0.0000 | 0.0000 | 0.0005 | 0.0013 | 0.0005 | | 0.62 | 1.00 | 0.21 |

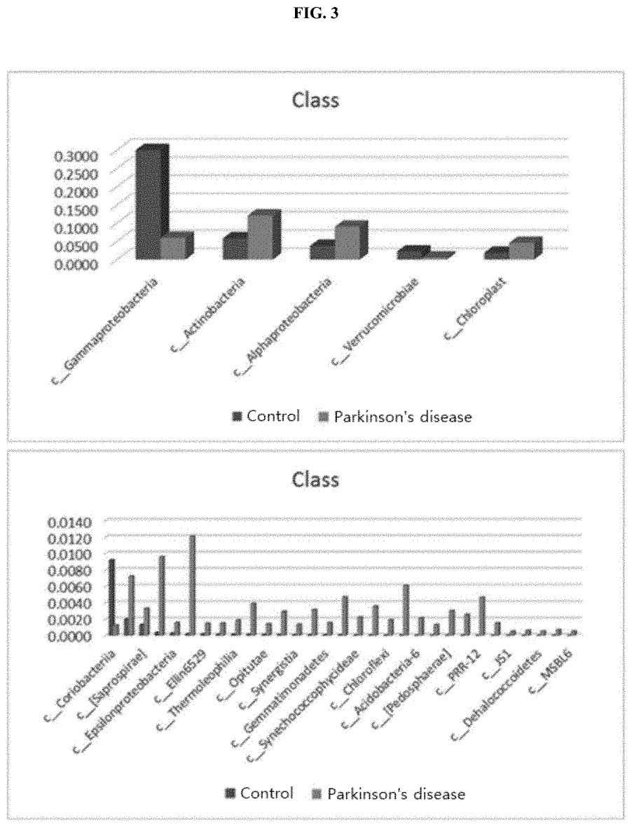

As a result of analyzing bacteria-derived EVs in urine at a class level, a diagnostic model developed using bacteria belonging to the class Coriobacteriia, the class Gammaproteobacteria, the class Verrucomicrobiae, the class Actinobacteria, the class Alphaproteobacteria, the class Chloroplast, the class Saprospirae, the class Deltaproteobacteria, the class Epsilonproteobacteria, the class Ellin6529, the class Chloracidobacteria, the class Opitutae, the class Thermoleophilia, the class Synergistia, the class Gemmatimonadetes, the class Planctomycetia, the class Acidobacteriia, the class Solibacteres, the class Anaerolineae, the class Chloroflexi, the class Phycisphaerae, the class Synechococcophycideae, the class TM7-1, the class Acidimicrobiia, the class Acidobacteria-6, the class Spartobacteria, the class ABY1, the class Pedosphaerae, the class ZB2, the class PRR-12, the class Ktedonobacteria, the class JS1, the class WM88, the class Dehalococcoidetes, the class SAR202, and the class MSBL6 as a biomarker exhibited significant diagnostic performance for Parkinson's disease (see Table 3 and FIG. 3).

TABLE 3

| | | | Parkinson's | | | | | | |
|---|---|---|---|---|---|---|---|---|---|
| | Control | | disease | | t-test | | | | |
| Taxon | Mean | SD | Mean | SD | p-value | Ratio | AUC | Sensitivity | Specificity |
| c_Coriobacteriia | 0.0092 | 0.0080 | 0.0013 | 0.0015 | 0.0000 | 0.14 | 0.85 | 0.78 | 0.72 |
| c_Gammaproteobacteria | 0.2999 | 0.1716 | 0.0593 | 0.1103 | 0.0000 | 0.20 | 0.96 | 0.96 | 0.97 |
| c_Verrucomicrobiae | 0.0218 | 0.0272 | 0.0052 | 0.0044 | 0.0000 | 0.24 | 0.73 | 0.79 | 0.36 |
| c_Actinobacteria | 0.0579 | 0.0320 | 0.1202 | 0.0553 | 0.0000 | 2.08 | 0.91 | 0.88 | 0.74 |
| c_Alphaproteobacteria | 0.0372 | 0.0434 | 0.0897 | 0.0244 | 0.0000 | 2.41 | 0.94 | 0.96 | 0.79 |
| c_Chloroplast | 0.0185 | 0.0202 | 0.0454 | 0.0181 | 0.0000 | 2.46 | 0.86 | 0.86 | 0.51 |
| c_[Saprospirae] | 0.0013 | 0.0030 | 0.0033 | 0.0030 | 0.0015 | 2.46 | 0.75 | 0.92 | 0.23 |
| c_Deltaproteobacteria | 0.0020 | 0.0036 | 0.0072 | 0.0040 | 0.0000 | 3.61 | 0.85 | 0.89 | 0.51 |
| c_Epsilonproteobacteria | 0.0003 | 0.0010 | 0.0016 | 0.0021 | 0.0007 | 5.52 | 0.74 | 0.95 | 0.44 |
| c_Ellin6529 | 0.0002 | 0.0016 | 0.0015 | 0.0016 | 0.0001 | 7.88 | 0.89 | 0.99 | 0.49 |
| c_[Chloracidobacteria] | 0.0001 | 0.0008 | 0.0015 | 0.0015 | 0.0000 | 10.81 | 0.87 | 0.97 | 0.54 |
| c_Opitutae | 0.0001 | 0.0006 | 0.0014 | 0.0020 | 0.0002 | 12.65 | 0.75 | 0.95 | 0.44 |
| c_Thermoleophilia | 0.0001 | 0.0006 | 0.0019 | 0.0018 | 0.0000 | 13.97 | 0.86 | 0.96 | 0.64 |
| c_Synergistia | 0.0001 | 0.0003 | 0.0014 | 0.0018 | 0.0001 | 15.89 | 0.81 | 0.96 | 0.49 |
| c_Gemmatimonadetes | 0.0001 | 0.0004 | 0.0016 | 0.0015 | 0.0000 | 21.15 | 0.82 | 0.96 | 0.64 |
| c_Planctomycetia | 0.0004 | 0.0015 | 0.0096 | 0.0049 | 0.0000 | 25.27 | 0.98 | 0.97 | 0.92 |
| c_Acidobacteriia | 0.0001 | 0.0005 | 0.0029 | 0.0024 | 0.0000 | 30.81 | 0.92 | 0.96 | 0.77 |
| c_Solibacteres | 0.0001 | 0.0006 | 0.0039 | 0.0036 | 0.0000 | 33.37 | 0.92 | 0.96 | 0.74 |
| c_Anaerolineae | 0.0001 | 0.0004 | 0.0032 | 0.0029 | 0.0000 | 40.61 | 0.89 | 0.97 | 0.69 |
| c_Chloroflexi | 0.0000 | 0.0002 | 0.0019 | 0.0018 | 0.0000 | 55.61 | 0.84 | 0.97 | 0.59 |
| c_Phycisphaerae | 0.0002 | 0.0013 | 0.0121 | 0.0077 | 0.0000 | 59.10 | 0.96 | 0.99 | 0.85 |
| c_Synechococcophycideae | 0.0000 | 0.0003 | 0.0022 | 0.0030 | 0.0000 | 61.69 | 0.86 | 0.99 | 0.49 |
| c_TM7-1 | 0.0001 | 0.0003 | 0.0047 | 0.0040 | 0.0000 | 89.44 | 0.94 | 0.99 | 0.77 |
| c_Acidimicrobiia | 0.0000 | 0.0003 | 0.0036 | 0.0025 | 0.0000 | 100.35 | 0.97 | 0.99 | 0.85 |
| c_Acidobacteria-6 | 0.0000 | 0.0001 | 0.0022 | 0.0021 | 0.0000 | 140.15 | 0.88 | 0.99 | 0.69 |
| c_[Spartobacteria] | 0.0000 | 0.0002 | 0.0061 | 0.0038 | 0.0000 | 201.78 | 0.99 | 0.99 | 0.92 |
| c_ABY1 | 0.0000 | 0.0000 | 0.0013 | 0.0022 | 0.0008 | 324.38 | 0.78 | 0.99 | 0.44 |
| c_[Pedosphaerae] | 0.0000 | 0.0000 | 0.0030 | 0.0019 | 0.0000 | 2033.19 | 1.00 | 1.00 | 1.00 |
| c_ZB2 | 0.0000 | 0.0000 | 0.0026 | 0.0034 | 0.0000 | 10532.52 | 0.90 | 0.99 | 0.72 |
| c_PRR-12 | 0.0000 | 0.0000 | 0.0046 | 0.0027 | 0.0000 | | 1.00 | 1.00 | 1.00 |
| c_Ktedonobacteria | 0.0000 | 0.0000 | 0.0015 | 0.0019 | 0.0000 | | 0.88 | 1.00 | 0.74 |
| c_JS1 | 0.0000 | 0.0000 | 0.0005 | 0.0009 | 0.0000 | | 0.76 | 1.00 | 0.49 |
| c_WM88 | 0.0000 | 0.0000 | 0.0006 | 0.0012 | 0.0000 | | 0.75 | 1.00 | 0.44 |
| c_Dehalococcoidetes | 0.0000 | 0.0000 | 0.0005 | 0.0012 | 0.0001 | | 0.73 | 1.00 | 0.41 |
| c_SAR202 | 0.0000 | 0.0000 | 0.0007 | 0.0015 | 0.0001 | | 0.72 | 1.00 | 0.36 |
| c_MSBL6 | 0.0000 | 0.0000 | 0.0005 | 0.0011 | 0.0000 | | 0.70 | 1.00 | 0.38 |

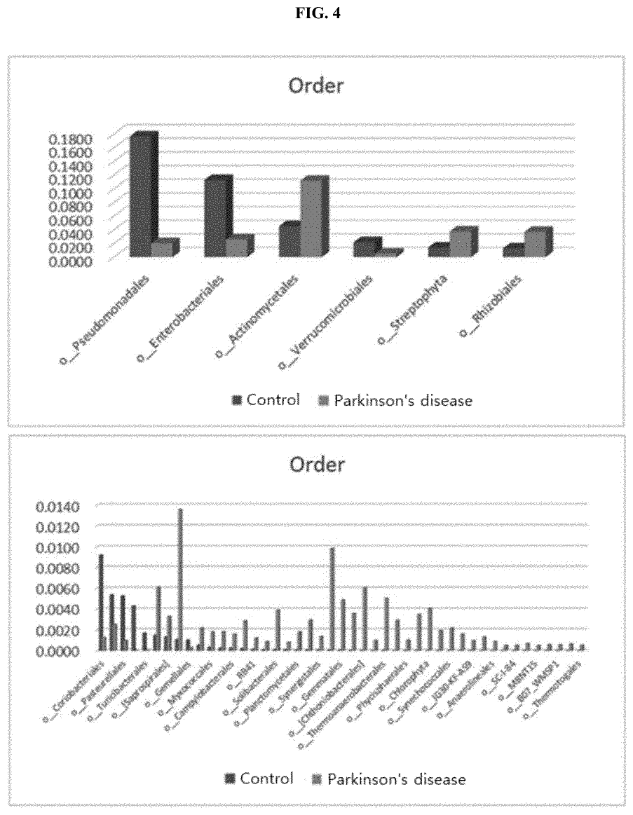

As a result of analyzing bacteria-derived EVs in urine at an order level, a diagnostic model developed using bacteria belonging to the order RF39, the order Turicibacterales, the order Pseudomonadales, the order Coriobacteriales, the order Pasteurellales, the order Enterobacteriales, the order Verrucomicrobiales, the order Gemellales, the order Neisseriales, the order Saprospirales, the order Actinomycetales, the order Streptophyta, the order Rhizobiales, the order Rhodospirillales, the order Xanthomonadales, the order Myxococcales, the order Campylobacterales, the order Desulfovibrionales, the order Solirubrobacterales, the order Opitutales, the order RB41, the order Rickettsiales, the order Pirellulales, the order Synergistales, the order Planctomycetales, the order Acidobacteriales, the order Solibacterales, the order Gaiellales, the order Gemmatales, the order Acidimicrobiales, the order WD2101, the order Chthoniobacterales, the order Thermoanaerobacterales, the order Pedosphaerales, the order Phycisphaerales, the order Sediment-1, the order Chlorophyta, the order iii1-15, the order Synechococcales, the order Roseiflexales, the order JG30-KF-AS9, the order Ellin329, the order Anaerolineales, the order Ellin5290, the order SC-I-84, the order Cryptophyta, the order MBNT15, the order envOPS12, the order B07_WMSP1, the order UA01, and the order Thermotogales as a biomarker exhibited significant diagnostic performance for Parkinson's disease (see Table 4 and FIG. 4).

TABLE 4

| | | | Parkinson's | | | | | | |
|---|---|---|---|---|---|---|---|---|---|
| | Control | | disease | | t-test | | | | |
| Taxon | Mean | SD | Mean | SD | p-value | Ratio | AUC | Sensitivity | Specificity |
| o_RF39 | 0.0043 | 0.0078 | 0.0000 | 0.0000 | 0.0000 | 0.00 | 0.93 | 0.86 | 0.97 |
| o_Turicibacterales | 0.0017 | 0.0025 | 0.0001 | 0.0004 | 0.0000 | 0.04 | 0.90 | 0.74 | 0.97 |
| o_Pseudomonadales | 0.1765 | 0.1640 | 0.0206 | 0.0091 | 0.0000 | 0.12 | 1.00 | 0.97 | 1.00 |
| o_Coriobacteriales | 0.0092 | 0.0080 | 0.0013 | 0.0015 | 0.0000 | 0.14 | 0.93 | 0.87 | 0.92 |
| o_Pasteurellales | 0.0053 | 0.0061 | 0.0010 | 0.0013 | 0.0000 | 0.19 | 0.91 | 0.82 | 0.79 |
| o_Enterobacteriales | 0.1129 | 0.0780 | 0.0260 | 0.1144 | 0.0001 | 0.23 | 0.98 | 0.93 | 0.97 |
| o_Verrucomicrobiales | 0.0218 | 0.0272 | 0.0052 | 0.0044 | 0.0000 | 0.24 | 0.90 | 0.82 | 0.90 |
| o_Gemellales | 0.0010 | 0.0017 | 0.0003 | 0.0008 | 0.0036 | 0.31 | 0.83 | 0.74 | 0.74 |
| o_Neisseriales | 0.0054 | 0.0077 | 0.0026 | 0.0026 | 0.0050 | 0.48 | 0.82 | 0.75 | 0.62 |
| o_[Saprospirales] | 0.0013 | 0.0030 | 0.0033 | 0.0030 | 0.0015 | 2.46 | 0.88 | 0.89 | 0.62 |
| o_Actinomycetales | 0.0452 | 0.0290 | 0.1116 | 0.0549 | 0.0000 | 2.47 | 0.95 | 0.89 | 0.90 |
| o_Streptophyta | 0.0142 | 0.0184 | 0.0375 | 0.0152 | 0.0000 | 2.63 | 0.93 | 0.91 | 0.82 |
| o_Rhizobiales | 0.0128 | 0.0111 | 0.0368 | 0.0148 | 0.0000 | 2.88 | 0.95 | 0.96 | 0.79 |
| o_Rhodospirillales | 0.0005 | 0.0017 | 0.0022 | 0.0022 | 0.0001 | 4.04 | 0.90 | 0.93 | 0.59 |
| o_Xanthomonadales | 0.0015 | 0.0022 | 0.0061 | 0.0036 | 0.0000 | 4.12 | 0.94 | 0.89 | 0.69 |
| o_Myxococcales | 0.0003 | 0.0013 | 0.0018 | 0.0022 | 0.0004 | 5.24 | 0.89 | 0.87 | 0.64 |
| o_Campylobacterales | 0.0003 | 0.0010 | 0.0016 | 0.0021 | 0.0007 | 5.52 | 0.88 | 0.91 | 0.56 |
| o_Desulfovibrionales | 0.0003 | 0.0007 | 0.0018 | 0.0022 | 0.0001 | 6.33 | 0.89 | 0.84 | 0.67 |
| o_Solirubrobacterales | 0.0001 | 0.0006 | 0.0009 | 0.0014 | 0.0027 | 7.42 | 0.85 | 0.87 | 0.62 |
| o_Opitutales | 0.0001 | 0.0005 | 0.0008 | 0.0015 | 0.0070 | 7.54 | 0.83 | 0.91 | 0.38 |
| o_RB41 | 0.0001 | 0.0008 | 0.0012 | 0.0014 | 0.0000 | 9.27 | 0.98 | 0.99 | 0.79 |
| o_Rickettsiales | 0.0011 | 0.0019 | 0.0136 | 0.0086 | 0.0000 | 12.59 | 0.98 | 0.97 | 0.90 |
| o_Pirellulales | 0.0002 | 0.0014 | 0.0029 | 0.0023 | 0.0000 | 13.12 | 0.93 | 0.99 | 0.72 |
| o_Synergistales | 0.0001 | 0.0003 | 0.0014 | 0.0018 | 0.0001 | 15.89 | 0.91 | 0.97 | 0.59 |
| o_Planctomycetales | 0.0001 | 0.0006 | 0.0018 | 0.0021 | 0.0000 | 18.78 | 0.91 | 0.95 | 0.59 |
| o_Acidobacteriales | 0.0001 | 0.0005 | 0.0029 | 0.0024 | 0.0000 | 30.81 | 0.96 | 0.97 | 0.82 |
| o_Solibacterales | 0.0001 | 0.0006 | 0.0039 | 0.0036 | 0.0000 | 33.37 | 0.96 | 0.99 | 0.74 |
| o_Gaiellales | 0.0000 | 0.0001 | 0.0010 | 0.0013 | 0.0000 | 61.75 | 0.91 | 0.99 | 0.54 |
| o_Gemmatales | 0.0001 | 0.0003 | 0.0049 | 0.0036 | 0.0000 | 76.01 | 0.97 | 0.99 | 0.79 |
| o_Acidimicrobiales | 0.0000 | 0.0003 | 0.0036 | 0.0025 | 0.0000 | 100.35 | 1.00 | 0.99 | 0.90 |
| o_WD2101 | 0.0001 | 0.0004 | 0.0098 | 0.0067 | 0.0000 | 153.20 | 0.99 | 0.99 | 0.92 |
| o_[Chthoniobacterales] | 0.0000 | 0.0002 | 0.0061 | 0.0038 | 0.0000 | 201.78 | 1.00 | 1.00 | 1.00 |
| o_Thermoanaerobacterales | 0.0000 | 0.0001 | 0.0050 | 0.0028 | 0.0000 | 337.41 | 0.99 | 0.99 | 0.92 |
| o_[Pedosphaerales] | 0.0000 | 0.0000 | 0.0029 | 0.0019 | 0.0000 | 1980.86 | 1.00 | 1.00 | 1.00 |
| o_Phycisphaerales | 0.0000 | 0.0000 | 0.0010 | 0.0022 | 0.0060 | 3096.39 | 0.89 | 0.99 | 0.49 |
| o_Sediment-1 | 0.0000 | 0.0000 | 0.0035 | 0.0023 | 0.0000 | | 1.00 | 1.00 | 0.97 |
| o_Chlorophyta | 0.0000 | 0.0000 | 0.0041 | 0.0047 | 0.0000 | | 0.98 | 1.00 | 0.92 |
| o_iii1-15 | 0.0000 | 0.0000 | 0.0019 | 0.0021 | 0.0000 | | 0.97 | 1.00 | 0.82 |
| o_Synechococcales | 0.0000 | 0.0000 | 0.0022 | 0.0029 | 0.0000 | | 0.95 | 1.00 | 0.79 |
| o_[Roseiflexales] | 0.0000 | 0.0000 | 0.0016 | 0.0016 | 0.0000 | | 0.95 | 1.00 | 0.69 |
| o_JG30-KF-AS9 | 0.0000 | 0.0000 | 0.0010 | 0.0013 | 0.0000 | | 0.93 | 1.00 | 0.67 |
| o_Ellin329 | 0.0000 | 0.0000 | 0.0013 | 0.0026 | 0.0000 | | 0.93 | 1.00 | 0.59 |
| o_Anaerolineales | 0.0000 | 0.0000 | 0.0009 | 0.0017 | 0.0000 | | 0.89 | 0.97 | 0.49 |
| o_Ellin5290 | 0.0000 | 0.0000 | 0.0005 | 0.0011 | 0.0000 | | 0.89 | 0.92 | 0.56 |
| o_SC-I-84 | 0.0000 | 0.0000 | 0.0005 | 0.0010 | 0.0000 | | 0.89 | 1.00 | 0.46 |
| o_Cryptophyta | 0.0000 | 0.0000 | 0.0007 | 0.0013 | 0.0000 | | 0.88 | 0.97 | 0.46 |
| o_MBNT15 | 0.0000 | 0.0000 | 0.0005 | 0.0011 | 0.0001 | | 0.88 | 0.92 | 0.59 |
| o_envOPS12 | 0.0000 | 0.0000 | 0.0006 | 0.0011 | 0.0000 | | 0.87 | 1.00 | 0.41 |
| o_B07_WMSP1 | 0.0000 | 0.0000 | 0.0006 | 0.0013 | 0.0003 | | 0.87 | 0.92 | 0.46 |
| o_UA01 | 0.0000 | 0.0000 | 0.0007 | 0.0020 | 0.0036 | | 0.86 | 0.97 | 0.38 |
| o_Thermotogales | 0.0000 | 0.0000 | 0.0005 | 0.0013 | 0.0005 | | 0.83 | 0.89 | 0.44 |

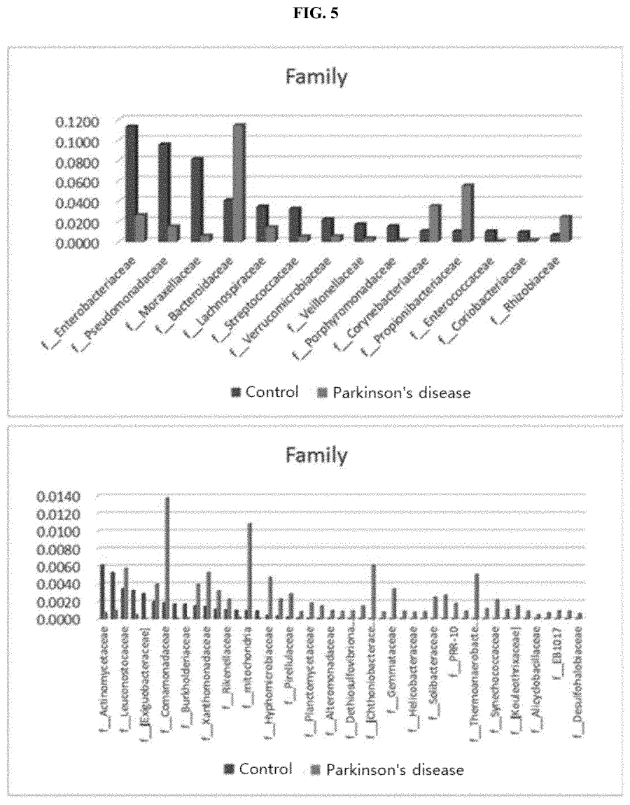

As a result of analyzing bacteria-derived EVs in urine at a family level, a diagnostic model developed using bacteria belonging to the family Exiguobacteraceae, the family Enterococcaceae, the family Turicibacteraceae, the family Mogibacteriaceae, the family Moraxellaceae, the family Porphyromonadaceae, the family Burkholderiaceae, the family Actinomycetaceae, the family Coriobacteriaceae, the family Methylobacteriaceae, the family Streptococcaceae, the family Pseudomonadaceae, the family Pasteurellaceae, the family Veillonellaceae, the family Peptostreptococcaceae, the family Enterobacteriaceae, the family Verrucomicrobiaceae, the family Lachnospiraceae, the family Leuconostocaceae, the family Bradyrhizobiaceae, the family Rikenellaceae, the family Tissierellaceae, the family Bacteroidaceae, the family Chitinophagaceae, the family Corynebacteriaceae, the family Xanthomonadaceae, the family Rhizobiaceae, the family Propionibacteriaceae, the family Desulfobacteraceae, the family Barnesiellaceae, the family Comamonadaceae, the family mitochondria, the family Hyphomicrobiaceae, the family Alteromonadaceae, the family Sinobacteraceae, the family Pirellulaceae, the family Dethiosulfovibrionaceae, the family Acidobacteriaceae, the family Planctomycetaceae, the family Isosphaeraceae, the family Gaiellaceae, the family Koribacteraceae, the family Helicobacteraceae, the family Chthoniobacteraceae, the family Gemmataceae, the family C111, the family Solibacteraceae, the family Pelagibacteraceae, the family PRR-10, the family Ellin515, the family Thermoanaerobacteraceae, the family Methanoregulaceae, the family Synechococcaceae, the family Desulfomicrobiaceae, the family Kouleothrixaceae, the family OCS155, the family Alicyclobacillaceae, the family Myxococcaceae, the family EB1017, the family Anaerolinaceae, and the family Desulfohalobiaceae as a biomarker exhibited significant diagnostic performance for Parkinson's disease (see Table 5 and FIG. 5).

TABLE 5

| | | | Parkinson's | | | | | | |
|---|---|---|---|---|---|---|---|---|---|
| | Control | | disease | | t-test | | | | |
| Taxon | Mean | SD | Mean | SD | p-value | Ratio | AUC | Sensitivity | Specificity |
| f_[Exiguobacteraceae] | 0.0029 | 0.0064 | 0.0000 | 0.0000 | 0.0002 | 0.00 | 0.73 | 0.87 | 0.21 |
| f_Enterococcaceae | 0.0100 | 0.0106 | 0.0003 | 0.0005 | 0.0000 | 0.03 | 0.92 | 0.87 | 0.90 |
| f_Turicibacteraceae | 0.0017 | 0.0025 | 0.0001 | 0.0004 | 0.0000 | 0.04 | 0.75 | 0.99 | 0.08 |
| f_[Mogibacteriaceae] | 0.0009 | 0.0014 | 0.0000 | 0.0003 | 0.0000 | 0.05 | 0.75 | 0.84 | 0.26 |
| f_Moraxellaceae | 0.0810 | 0.1062 | 0.0058 | 0.0041 | 0.0000 | 0.07 | 0.97 | 0.93 | 0.92 |
| f_Porphyromonadaceae | 0.0151 | 0.0169 | 0.0012 | 0.0017 | 0.0000 | 0.08 | 0.85 | 0.82 | 0.74 |
| f_Burkholderiaceae | 0.0017 | 0.0028 | 0.0001 | 0.0004 | 0.0000 | 0.09 | 0.79 | 0.91 | 0.44 |
| f_Actinomycetaceae | 0.0061 | 0.0095 | 0.0007 | 0.0010 | 0.0000 | 0.12 | 0.80 | 0.78 | 0.67 |
| f_Coriobacteriaceae | 0.0092 | 0.0080 | 0.0013 | 0.0015 | 0.0000 | 0.14 | 0.85 | 0.78 | 0.72 |
| f_Methylobacteriaceae | 0.0032 | 0.0056 | 0.0005 | 0.0009 | 0.0001 | 0.15 | 0.71 | 0.95 | 0.05 |
| f_Streptococcaceae | 0.0322 | 0.0227 | 0.0050 | 0.0035 | 0.0000 | 0.15 | 0.90 | 0.82 | 0.87 |
| f_Pseudomonadaceae | 0.0954 | 0.0850 | 0.0148 | 0.0074 | 0.0000 | 0.15 | 0.98 | 0.95 | 0.90 |
| f_Pasteurellaceae | 0.0053 | 0.0061 | 0.0010 | 0.0013 | 0.0000 | 0.19 | 0.75 | 0.72 | 0.59 |
| f_Veillonellaceae | 0.0169 | 0.0227 | 0.0032 | 0.0026 | 0.0000 | 0.19 | 0.79 | 0.78 | 0.69 |
| f_Peptostreptococcaceae | 0.0010 | 0.0015 | 0.0002 | 0.0006 | 0.0001 | 0.20 | 0.73 | 0.97 | 0.05 |
| f_Enterobacteriaceae | 0.1129 | 0.0780 | 0.0260 | 0.1144 | 0.0001 | 0.23 | 0.94 | 0.95 | 0.95 |
| f_Verrucomicrobiaceae | 0.0218 | 0.0272 | 0.0052 | 0.0044 | 0.0000 | 0.24 | 0.73 | 0.79 | 0.36 |
| f_Lachnospiraceae | 0.0343 | 0.0180 | 0.0140 | 0.0071 | 0.0000 | 0.41 | 0.89 | 0.88 | 0.74 |
| f_Leuconostocaceae | 0.0034 | 0.0039 | 0.0057 | 0.0035 | 0.0030 | 1.67 | 0.72 | 0.93 | 0.13 |
| f_Bradyrhizobiaceae | 0.0020 | 0.0050 | 0.0039 | 0.0034 | 0.0170 | 1.97 | 0.74 | 0.96 | 0.10 |
| f_Rikenellaceae | 0.0011 | 0.0021 | 0.0022 | 0.0023 | 0.0069 | 2.11 | 0.74 | 0.93 | 0.13 |
| f_[Tissierellaceae] | 0.0015 | 0.0025 | 0.0039 | 0.0028 | 0.0000 | 2.63 | 0.78 | 0.89 | 0.38 |
| f_Bacteroidaceae | 0.0405 | 0.0357 | 0.1142 | 0.0288 | 0.0000 | 2.82 | 0.94 | 0.95 | 0.87 |
| f_Chitinophagaceae | 0.0011 | 0.0021 | 0.0032 | 0.0031 | 0.0004 | 2.89 | 0.76 | 0.93 | 0.31 |
| f_Corynebacteriaceae | 0.0103 | 0.0104 | 0.0348 | 0.0154 | 0.0000 | 3.37 | 0.92 | 0.89 | 0.72 |
| f_Xanthomonadaceae | 0.0014 | 0.0022 | 0.0052 | 0.0034 | 0.0000 | 3.70 | 0.85 | 0.86 | 0.54 |
| f_Rhizobiaceae | 0.0063 | 0.0064 | 0.0240 | 0.0107 | 0.0000 | 3.80 | 0.93 | 0.95 | 0.72 |
| f_Propionibacteriaceae | 0.0101 | 0.0087 | 0.0550 | 0.0434 | 0.0000 | 5.45 | 0.98 | 0.96 | 0.92 |
| f_Desulfobacteraceae | 0.0001 | 0.0007 | 0.0008 | 0.0013 | 0.0025 | 6.30 | 0.71 | 0.96 | 0.26 |
| f_[Barnesiellaceae] | 0.0003 | 0.0009 | 0.0023 | 0.0023 | 0.0000 | 6.56 | 0.81 | 0.93 | 0.51 |
| f_Comamonadaceae | 0.0019 | 0.0026 | 0.0137 | 0.0076 | 0.0000 | 7.40 | 0.98 | 0.96 | 0.79 |
| f_mitochondria | 0.0010 | 0.0018 | 0.0108 | 0.0073 | 0.0000 | 11.19 | 0.95 | 0.97 | 0.79 |
| f_Hyphomicrobiaceae | 0.0004 | 0.0016 | 0.0047 | 0.0046 | 0.0000 | 11.33 | 0.86 | 0.96 | 0.51 |
| f_Alteromonadaceae | 0.0001 | 0.0005 | 0.0010 | 0.0014 | 0.0008 | 12.22 | 0.77 | 0.97 | 0.36 |
| f_Sinobacteraceae | 0.0001 | 0.0003 | 0.0009 | 0.0014 | 0.0008 | 12.62 | 0.73 | 0.96 | 0.38 |
| f_Pirellulaceae | 0.0002 | 0.0014 | 0.0029 | 0.0023 | 0.0000 | 13.12 | 0.90 | 0.97 | 0.69 |
| f_Dethiosulfovibrionaceae | 0.0001 | 0.0003 | 0.0009 | 0.0014 | 0.0004 | 15.62 | 0.76 | 0.96 | 0.36 |
| f_Acidobacteriaceae | 0.0001 | 0.0005 | 0.0015 | 0.0020 | 0.0001 | 17.35 | 0.79 | 0.97 | 0.46 |
| f_Planctomycetaceae | 0.0001 | 0.0006 | 0.0018 | 0.0021 | 0.0000 | 18.78 | 0.82 | 0.97 | 0.49 |
| f_Isosphaeraceae | 0.0001 | 0.0003 | 0.0015 | 0.0019 | 0.0000 | 28.59 | 0.82 | 0.97 | 0.44 |
| f_Gaiellaceae | 0.0000 | 0.0001 | 0.0008 | 0.0010 | 0.0000 | 48.28 | 0.76 | 0.97 | 0.46 |
| f_Koribacteraceae | 0.0000 | 0.0001 | 0.0009 | 0.0015 | 0.0006 | 91.19 | 0.71 | 0.99 | 0.33 |
| f_Helicobacteraceae | 0.0000 | 0.0001 | 0.0008 | 0.0013 | 0.0015 | 114.37 | 0.84 | 0.99 | 0.41 |
| f_[Chthoniobacteraceae] | 0.0000 | 0.0002 | 0.0061 | 0.0038 | 0.0000 | 201.78 | 0.99 | 0.99 | 0.92 |
| f_Gemmataceae | 0.0000 | 0.0001 | 0.0034 | 0.0034 | 0.0000 | 273.92 | 0.91 | 0.99 | 0.69 |
| f_C111 | 0.0000 | 0.0000 | 0.0008 | 0.0013 | 0.0005 | 964.09 | 0.73 | 0.99 | 0.33 |
| f_Solibacteraceae | 0.0000 | 0.0000 | 0.0025 | 0.0024 | 0.0000 | 13883.24 | 0.95 | 0.99 | 0.90 |
| f_Pelagibacteraceae | 0.0000 | 0.0000 | 0.0027 | 0.0033 | 0.0000 | | 0.97 | 1.00 | 0.92 |
| f_PRR-10 | 0.0000 | 0.0000 | 0.0018 | 0.0017 | 0.0000 | | 0.96 | 1.00 | 0.90 |
| f_Ellin515 | 0.0000 | 0.0000 | 0.0009 | 0.0008 | 0.0000 | | 0.95 | 1.00 | 0.85 |
| f_Thermoanaerobacteraceae | 0.0000 | 0.0000 | 0.0050 | 0.0028 | 0.0000 | | 0.93 | 0.91 | 0.95 |
| f_Methanoregulaceae | 0.0000 | 0.0000 | 0.0012 | 0.0019 | 0.0000 | | 0.92 | 1.00 | 0.82 |
| f_Synechococcaceae | 0.0000 | 0.0000 | 0.0022 | 0.0029 | 0.0000 | | 0.91 | 1.00 | 0.79 |
| f_Desulfomicrobiaceae | 0.0000 | 0.0000 | 0.0011 | 0.0014 | 0.0000 | | 0.88 | 1.00 | 0.69 |
| f_[Kouleothrixaceae] | 0.0000 | 0.0000 | 0.0015 | 0.0015 | 0.0000 | | 0.86 | 1.00 | 0.67 |
| f_OCS155 | 0.0000 | 0.0000 | 0.0009 | 0.0015 | 0.0000 | | 0.84 | 1.00 | 0.62 |
| f_Alicyclobacillaceae | 0.0000 | 0.0000 | 0.0005 | 0.0009 | 0.0000 | | 0.79 | 1.00 | 0.49 |
| f_Myxococcaceae | 0.0000 | 0.0000 | 0.0007 | 0.0012 | 0.0000 | | 0.76 | 1.00 | 0.46 |
| f_EB1017 | 0.0000 | 0.0000 | 0.0009 | 0.0015 | 0.0000 | | 0.76 | 1.00 | 0.51 |
| f_Anaerolinaceae | 0.0000 | 0.0000 | 0.0009 | 0.0017 | 0.0000 | | 0.74 | 1.00 | 0.44 |
| f_Desulfohalobiaceae | 0.0000 | 0.0000 | 0.0006 | 0.0014 | 0.0002 | | 0.73 | 1.00 | 0.41 |

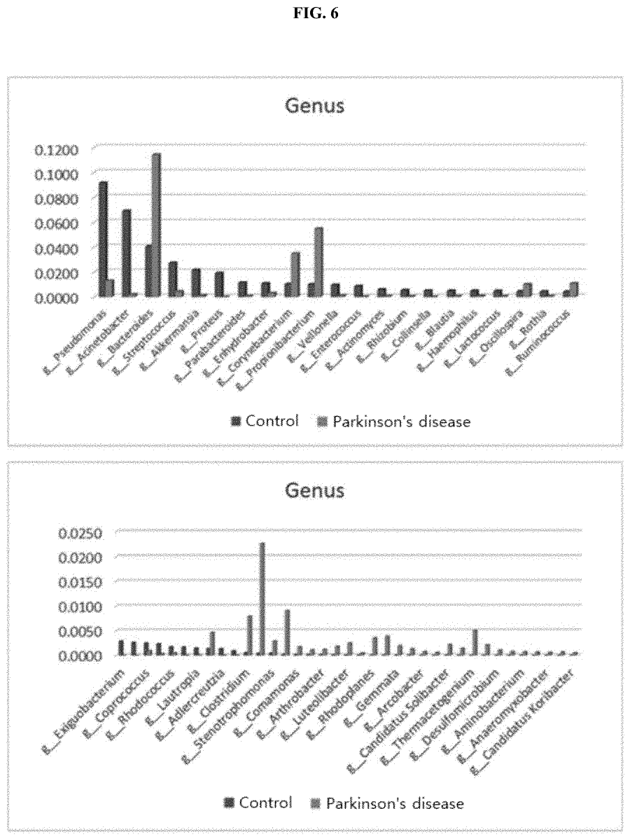

As a result of analyzing bacteria-derived EVs in urine at a genus level, a diagnostic model developed using bacteria belonging to the genus Collinsella, the genus Adlercreutzia, the genus SMB53, the genus Proteus, the genus Exiguobacterium, the genus Enterococcus, the genus Acinetobacter, the genus Turicibacter, the genus Klebsiella, the genus Lautropia, the genus Akkermansia, the genus Parabacteroides, the genus Rhizobium, the genus Actinomyces, the genus Lactococcus, the genus Blautia, the genus Veillonella, the genus Pseudomonas, the genus Rothia, the genus Dorea, the genus Streptococcus, the genus Haemophilus, the genus Enhydrobacter, the genus Rhodococcus, the genus Coprococcus, the genus Oscillospira, the genus Ruminococcus, the genus Bacteroides, the genus Corynebacterium, the genus Weissella, the genus Propionibacterium, the genus Lysinibacillus, the genus Stenotrophomonas, the genus Arthrobacter, the genus Comamonas, the genus Marinobacter, the genus Clostridium, the genus Planctomyces, the genus Luteolibacter, the genus Delftia, the genus Agrobacterium, the genus Rhodoplanes, the genus DA101, the genus Gemmata, the genus Coprobacillus, the genus Arcobacter, the genus Helicobacter, the genus Candidatus Solibacter, the genus Methanosarcina, the genus Thermacetogenium, the genus Synechococcus, the genus Desulfomicrobium, the genus Chthoniobacter, the genus Aminobacterium, the genus Gallicola, the genus Anaeromyxobacter, the genus Muricauda, and the genus Candidatus Koribacter as a biomarker exhibited significant diagnostic performance for Parkinson's disease (see Table 6 and FIG. 6).

TABLE 6

| | | | Parkinson's | | | | | | |
|---|---|---|---|---|---|---|---|---|---|
| | Control | | disease | | t-test | | | | |
| Taxon | Mean | SD | Mean | SD | p-value | Ratio | AUC | Sensitivity | Specificity |
| g_Collinsella | 0.0050 | 0.0066 | 0.0000 | 0.0000 | 0.0000 | 0.00 | 0.88 | 0.74 | 1.00 |
| g_Adlercreutzia | 0.0013 | 0.0021 | 0.0000 | 0.0000 | 0.0001 | 0.00 | 0.80 | 0.59 | 1.00 |
| g_SMB53 | 0.0026 | 0.0037 | 0.0000 | 0.0000 | 0.0000 | 0.00 | 0.83 | 0.68 | 0.87 |
| g_Proteus | 0.0192 | 0.0298 | 0.0000 | 0.0000 | 0.0000 | 0.00 | 0.97 | 0.91 | 1.00 |
| g_Exiguobacterium | 0.0029 | 0.0064 | 0.0000 | 0.0000 | 0.0002 | 0.00 | 0.74 | 0.87 | 0.23 |
| g_Enterococcus | 0.0086 | 0.0099 | 0.0001 | 0.0003 | 0.0000 | 0.02 | 0.93 | 0.84 | 0.92 |
| g_Acinetobacter | 0.0690 | 0.1047 | 0.0020 | 0.0022 | 0.0000 | 0.03 | 0.97 | 0.93 | 0.97 |
| g_Turicibacter | 0.0017 | 0.0025 | 0.0001 | 0.0004 | 0.0000 | 0.04 | 0.75 | 0.99 | 0.08 |
| g_Klebsiella | 0.0009 | 0.0016 | 0.0000 | 0.0002 | 0.0000 | 0.04 | 0.70 | 0.97 | 0.10 |
| g_Lautropia | 0.0015 | 0.0028 | 0.0001 | 0.0003 | 0.0000 | 0.05 | 0.75 | 0.92 | 0.26 |
| g_Akkermansia | 0.0216 | 0.0271 | 0.0012 | 0.0025 | 0.0000 | 0.06 | 0.88 | 0.82 | 0.82 |
| g_Parabacteroides | 0.0115 | 0.0156 | 0.0007 | 0.0015 | 0.0000 | 0.06 | 0.84 | 0.75 | 0.82 |
| g_Rhizobium | 0.0054 | 0.0061 | 0.0004 | 0.0014 | 0.0000 | 0.07 | 0.93 | 0.84 | 0.92 |
| g_Actinomyces | 0.0059 | 0.0095 | 0.0005 | 0.0008 | 0.0000 | 0.09 | 0.83 | 0.78 | 0.69 |
| g_Lactococcus | 0.0048 | 0.0064 | 0.0005 | 0.0009 | 0.0000 | 0.10 | 0.76 | 0.75 | 0.56 |
| g_Blautia | 0.0049 | 0.0067 | 0.0005 | 0.0009 | 0.0000 | 0.10 | 0.80 | 0.70 | 0.72 |
| g_Veillonella | 0.0096 | 0.0188 | 0.0010 | 0.0014 | 0.0002 | 0.11 | 0.78 | 0.75 | 0.62 |
| g_Pseudomonas | 0.0916 | 0.0826 | 0.0129 | 0.0070 | 0.0000 | 0.14 | 0.98 | 0.95 | 0.92 |
| g_Rothia | 0.0043 | 0.0058 | 0.0007 | 0.0014 | 0.0000 | 0.15 | 0.73 | 0.76 | 0.46 |
| g_Dorea | 0.0023 | 0.0037 | 0.0004 | 0.0011 | 0.0000 | 0.15 | 0.75 | 0.93 | 0.21 |
| g_Streptococcus | 0.0272 | 0.0210 | 0.0044 | 0.0033 | 0.0000 | 0.16 | 0.89 | 0.83 | 0.85 |
| g_Haemophilus | 0.0049 | 0.0059 | 0.0009 | 0.0011 | 0.0000 | 0.18 | 0.75 | 0.71 | 0.56 |
| g_Enhydrobacter | 0.0108 | 0.0104 | 0.0031 | 0.0036 | 0.0000 | 0.28 | 0.78 | 0.84 | 0.56 |
| g_Rhodococcus | 0.0018 | 0.0028 | 0.0006 | 0.0012 | 0.0018 | 0.32 | 0.72 | 0.99 | 0.10 |
| g_Coprococcus | 0.0025 | 0.0026 | 0.0009 | 0.0017 | 0.0002 | 0.37 | 0.75 | 0.97 | 0.15 |
| g_Oscillospira | 0.0045 | 0.0048 | 0.0101 | 0.0055 | 0.0000 | 2.24 | 0.81 | 0.89 | 0.44 |
| g_Ruminococcus | 0.0041 | 0.0042 | 0.0108 | 0.0058 | 0.0000 | 2.62 | 0.83 | 0.89 | 0.51 |
| g_Bacteroides | 0.0405 | 0.0357 | 0.1142 | 0.0288 | 0.0000 | 2.82 | 0.94 | 0.95 | 0.87 |
| g_Corynebacterium | 0.0103 | 0.0104 | 0.0348 | 0.0154 | 0.0000 | 3.37 | 0.92 | 0.89 | 0.72 |
| g_Weissella | 0.0014 | 0.0020 | 0.0046 | 0.0034 | 0.0000 | 3.37 | 0.85 | 0.91 | 0.62 |
| g_Propionibacterium | 0.0101 | 0.0087 | 0.0548 | 0.0434 | 0.0000 | 5.43 | 0.98 | 0.96 | 0.92 |
| g_Lysinibacillus | 0.0002 | 0.0011 | 0.0011 | 0.0014 | 0.0008 | 5.46 | 0.78 | 0.95 | 0.46 |
| g_Stenotrophomonas | 0.0005 | 0.0012 | 0.0029 | 0.0029 | 0.0000 | 6.05 | 0.87 | 0.93 | 0.46 |
| g_Arthrobacter | 0.0002 | 0.0011 | 0.0012 | 0.0019 | 0.0028 | 6.37 | 0.73 | 0.97 | 0.28 |
| g_Comamonas | 0.0002 | 0.0007 | 0.0017 | 0.0016 | 0.0000 | 7.76 | 0.83 | 0.95 | 0.51 |
| g_Marinobacter | 0.0001 | 0.0005 | 0.0005 | 0.0008 | 0.0030 | 8.31 | 0.71 | 0.99 | 0.31 |
| g_Clostridium | 0.0006 | 0.0012 | 0.0079 | 0.0055 | 0.0000 | 13.95 | 0.95 | 0.93 | 0.77 |
| g_Planctomyces | 0.0001 | 0.0006 | 0.0018 | 0.0021 | 0.0000 | 18.78 | 0.82 | 0.97 | 0.49 |
| g_Luteolibacter | 0.0001 | 0.0003 | 0.0025 | 0.0025 | 0.0000 | 32.91 | 0.96 | 0.96 | 0.74 |
| g_Delftia | 0.0002 | 0.0011 | 0.0091 | 0.0063 | 0.0000 | 39.85 | 0.99 | 0.97 | 0.90 |
| g_Agrobacterium | 0.0005 | 0.0011 | 0.0226 | 0.0108 | 0.0000 | 45.62 | 1.00 | 0.99 | 0.95 |
| g_Rhodoplanes | 0.0000 | 0.0001 | 0.0036 | 0.0040 | 0.0000 | 120.50 | 0.86 | 0.99 | 0.62 |
| g_DA101 | 0.0000 | 0.0002 | 0.0039 | 0.0028 | 0.0000 | 153.39 | 0.97 | 0.99 | 0.79 |
| g_Gemmata | 0.0000 | 0.0001 | 0.0019 | 0.0028 | 0.0002 | 157.09 | 0.80 | 0.99 | 0.46 |
| g_Coprobacillus | 0.0000 | 0.0000 | 0.0014 | 0.0015 | 0.0000 | 246.64 | 0.88 | 0.99 | 0.62 |
| g_Arcobacter | 0.0000 | 0.0000 | 0.0008 | 0.0015 | 0.0031 | 335.36 | 0.75 | 0.99 | 0.33 |
| g_Helicobacter | 0.0000 | 0.0000 | 0.0006 | 0.0013 | 0.0072 | 770.90 | 0.70 | 0.99 | 0.28 |
| g_Candidatus | 0.0000 | 0.0000 | 0.0022 | 0.0024 | 0.0000 | 12365.58 | 0.89 | 0.99 | 0.77 |
| Solibacter | | | | | | | | | |
| g_Methanosarcina | 0.0000 | 0.0000 | 0.0014 | 0.0022 | 0.0000 | | 0.98 | 1.00 | 0.90 |
| g_Thermacetogenium | 0.0000 | 0.0000 | 0.0050 | 0.0028 | 0.0000 | | 0.93 | 0.91 | 0.95 |
| g_Synechococcus | 0.0000 | 0.0000 | 0.0021 | 0.0027 | 0.0000 | | 0.91 | 1.00 | 0.79 |
| g_Desulfomicrobium | 0.0000 | 0.0000 | 0.0011 | 0.0014 | 0.0000 | | 0.88 | 1.00 | 0.69 |
| g_Chthoniobacter | 0.0000 | 0.0000 | 0.0008 | 0.0012 | 0.0000 | | 0.88 | 1.00 | 0.69 |
| g_Aminobacterium | 0.0000 | 0.0000 | 0.0008 | 0.0012 | 0.0000 | | 0.87 | 1.00 | 0.69 |
| g_Gallicola | 0.0000 | 0.0000 | 0.0007 | 0.0012 | 0.0000 | | 0.76 | 1.00 | 0.44 |
| g_Anaeromyxobacter | 0.0000 | 0.0000 | 0.0006 | 0.0011 | 0.0000 | | 0.75 | 1.00 | 0.44 |
| g_Muricauda | 0.0000 | 0.0000 | 0.0007 | 0.0014 | 0.0000 | | 0.74 | 1.00 | 0.46 |
| g_Candidatus | 0.0000 | 0.0000 | 0.0005 | 0.0011 | 0.0001 | | 0.72 | 1.00 | 0.41 |
| Koribacter | | | | | | | | | |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of providing information for Parkinson's disease diagnosis through bacterial metagenomic analysis, according to the present invention, can be used to predict a risk of developing Parkinson's disease and diagnose Parkinson's disease by analyzing an increase or decrease in content of extracellular vesicles derived from specific bacteria through bacterial metagenomic analysis using a subject-derived sample. Extracellular vesicles secreted from microorganisms such as bacteria, archaea, and the like present in the environment are absorbed into the human body and distributed in the brain, and thus can directly affect inflammatory responses and brain functions, and since it is difficult to implement early diagnosis for Parkinson's disease, which is characterized by inflammation, before symptoms appear, efficient treatment is difficult. Thus, according to the present invention, a risk for developing Parkinson's disease may be predicted through metagenomic analysis of bacteria-derived extracellular vesicles using a human body-derived sample, and thus the onset of Parkinson's disease can be delayed or prevented through appropriate management by early diagnosis and prediction of a risk group for Parkinson's disease, and, even after Parkinson's disease occurs, early diagnosis for Parkinson's disease can be implemented, thereby lowering the incidence rate of Parkinson's disease and increasing therapeutic effects. In addition, patients diagnosed with Parkinson's disease are able to avoid exposure to causative factors predicted through bacterial metagenomic analysis according to the present invention, whereby the progression of Parkinson's disease is ameliorated, or recurrence thereof can be prevented.

1. A method of providing information for Parkinson's disease diagnosis, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

2. The method of claim 1, wherein process (c) comprises comparing an increase or decrease in content of extracellular vesicles derived from one or more bacteria selected from the group consisting of the phylum Proteobacteria, the phylum Cyanobacteria, the phylum Gemmatimonadetes, the phylum Chloroflexi, the phylum Synergistetes, the phylum Acidobacteria, the phylum Planctomycetes, the phylum OD1, the phylum WS3, the phylum Parvarchaeota, the phylum OP1, the phylum Chlorobi, the phylum OP9, the phylum Hyd24-12, and the phylum Thermotogae.

3. The method of claim 1, wherein process (c) comprises comparing an increase or decrease in content of extracellular vesicles derived from one or more bacteria selected from the group consisting of the class Coriobacteriia, the class Gammaproteobacteria, the class Verrucomicrobiae, the class Actinobacteria, the class Alphaproteobacteria, the class Chloroplast, the class Saprospirae, the class Deltaproteobacteria, the class Epsilonproteobacteria, the class Ellin6529, the class Chloracidobacteria, the class Opitutae, the class Thermoleophilia, the class Synergistia, the class Gemmatimonadetes, the class Planctomycetia, the class Acidobacteriia, the class Solibacteres, the class Anaerolineae, the class Chloroflexi, the class Phycisphaerae, the class Synechococcophycideae, the class TM7-1, the class Acidimicrobiia, the class Acidobacteria-6, the class Spartobacteria, the class ABY1, the class Pedosphaerae, the class ZB2, the class PRR-12, the class Ktedonobacteria, the class JS1, the class WM88, the class Dehalococcoidetes, the class SAR202, and the class MSBL6.

4. The method of claim 1, wherein process (c) comprises comparing an increase or decrease in content of extracellular vesicles derived from one or more bacteria selected from the group consisting of the order RF39, the order Turicibacterales, the order Pseudomonadales, the order Coriobacteriales, the order Pasteurellales, the order Enterobacteriales, the order Verrucomicrobiales, the order Gemellales, the order Neisseriales, the order Saprospirales, the order Actinomycetales, the order Streptophyta, the order Rhizobiales, the order Rhodospirillales, the order Xanthomonadales, the order Myxococcales, the order Campylobacterales, the order Desulfovibrionales, the order Solirubrobacterales, the order Opitutales, the order RB41, the order Rickettsiales, the order Pirellulales, the order Synergistales, the order Planctomycetales, the order Acidobacteriales, the order Solibacterales, the order Gaiellales, the order Gemmatales, the order Acidimicrobiales, the order WD2101, the order Chthoniobacterales, the order Thermoanaerobacterales, the order Pedosphaerales, the order Phycisphaerales, the order Sediment-1, the order Chlorophyta, the order iii1-15, the order Synechococcales, the order Roseiflexales, the order JG30-KF-AS9, the order Ellin329, the order Anaerolineales, the order Ellin5290, the order SC-I-84, the order Cryptophyta, the order MBNT15, the order envOPS12, the order B07_WMSP1, the order UA01, and the order Thermotogales.

5. The method of claim 1, wherein process (c) comprises comparing an increase or decrease in content of extracellular vesicles derived from one or more bacteria selected from the group consisting of the family Exiguobacteraceae, the family Enterococcaceae, the family Turicibacteraceae, the family Mogibacteriaceae, the family Moraxellaceae, the family Porphyromonadaceae, the family Burkholderiaceae, the family Actinomycetaceae, the family Coriobacteriaceae, the family Methylobacteriaceae, the family Streptococcaceae, the family Pseudomonadaceae, the family Pasteurellaceae, the family Veillonellaceae, the family Peptostreptococcaceae, the family Enterobacteriaceae, the family Verrucomicrobiaceae, the family Lachnospiraceae, the family Leuconostocaceae, the family Bradyrhizobiaceae, the family Rikenellaceae, the family Tissierellaceae, the family Bacteroidaceae, the family Chitinophagaceae, the family Corynebacteriaceae, the family Xanthomonadaceae, the family Rhizobiaceae, the family Propionibacteriaceae, the family Desulfobacteraceae, the family Barnesiellaceae, the family Comamonadaceae, the family mitochondria, the family Hyphomicrobiaceae, the family Alteromonadaceae, the family Sinobacteraceae, the family Pirellulaceae, the family Dethiosulfovibrionaceae, the family Acidobacteriaceae, the family Planctomycetaceae, the family Isosphaeraceae, the family Gaiellaceae, the family Koribacteraceae, the family Helicobacteraceae, the family Chthoniobacteraceae, the family Gemmataceae, the family C111, the family Solibacteraceae, the family Pelagibacteraceae, the family PRR-10, the family Ellin515, the family Thermoanaerobacteraceae, the family Methanoregulaceae, the family Synechococcaceae, the family Desulfomicrobiaceae, the family Kouleothrixaceae, the family OCS155, the family Alicyclobacillaceae, the family Myxococcaceae, the family EB1017, the family Anaerolinaceae, and the family Desulfohalobiaceae.

6. The method of claim 1, wherein process (c) comprises comparing an increase or decrease in content of extracellular vesicles derived from one or more bacteria selected from the group consisting of the genus Collinsella, the genus Adlercreutzia, the genus SMB53, the genus Proteus, the genus Exiguobacterium, the genus Enterococcus, the genus Acinetobacter, the genus Turicibacter, the genus Klebsiella, the genus Lautropia, the genus Akkermansia, the genus Parabacteroides, the genus Rhizobium, the genus Actinomyces, the genus Lactococcus, the genus Blautia, the genus Veillonella, the genus Pseudomonas, the genus Rothia, the genus Dorea, the genus Streptococcus, the genus Haemophilus, the genus Enhydrobacter, the genus Rhodococcus, the genus Coprococcus, the genus Oscillospira, the genus Ruminococcus, the genus Bacteroides, the genus Corynebacterium, the genus Weissella, the genus Propionibacterium, the genus Lysinibacillus, the genus Stenotrophomonas, the genus Arthrobacter, the genus Comamonas, the genus Marinobacter, the genus Clostridium, the genus Planctomyces, the genus Luteolibacter, the genus Delftia, the genus Agrobacterium, the genus Rhodoplanes, the genus DA101, the genus Gemmata, the genus Coprobacillus, the genus Arcobacter, the genus Helicobacter, the genus Candidatus Solibacter, the genus Methanosarcina, the genus Thermacetogenium, the genus Synechococcus, the genus Desulfomicrobium, the genus Chthoniobacter, the genus Aminobacterium, the genus Gallicola, the genus Anaeromyxobacter, the genus Muricauda, and the genus Candidatus Koribacter.

7. The method of claim 1, wherein the subject sample is urine.

8. A method of diagnosing Parkinson's disease, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

9. The method of claim 8, wherein process (c) comprises comparing an increase or decrease in content of extracellular vesicles derived from one or more bacteria selected from the group consisting of the phylum Proteobacteria, the phylum Cyanobacteria, the phylum Gemmatimonadetes, the phylum Chloroflexi, the phylum Synergistetes, the phylum Acidobacteria, the phylum Planctomycetes, the phylum OD1, the phylum WS3, the phylum Parvarchaeota, the phylum OP1, the phylum Chlorobi, the phylum OP9, the phylum Hyd24-12, and the phylum Thermotogae.

10. The method of claim 8, wherein process (c) comprises comparing an increase or decrease in content of extracellular vesicles derived from one or more bacteria selected from the group consisting of the class Coriobacteriia, the class Gammaproteobacteria, the class Verrucomicrobiae, the class Actinobacteria, the class Alphaproteobacteria, the class Chloroplast, the class Saprospirae, the class Deltaproteobacteria, the class Epsilonproteobacteria, the class Ellin6529, the class Chloracidobacteria, the class Opitutae, the class Thermoleophilia, the class Synergistia, the class Gemmatimonadetes, the class Planctomycetia, the class Acidobacteriia, the class Solibacteres, the class Anaerolineae, the class Chloroflexi, the class Phycisphaerae, the class Synechococcophycideae, the class TM7-1, the class Acidimicrobiia, the class Acidobacteria-6, the class Spartobacteria, the class ABY1, the class Pedosphaerae, the class ZB2, the class PRR-12, the class Ktedonobacteria, the class JS1, the class WM88, the class Dehalococcoidetes, the class SAR202, and the class MSBL6.

11. The method of claim 8, wherein process (c) comprises comparing an increase or decrease in content of extracellular vesicles derived from one or more bacteria selected from the group consisting of the order RF39, the order Turicibacterales, the order Pseudomonadales, the order Coriobacteriales, the order Pasteurellales, the order Enterobacteriales, the order Verrucomicrobiales, the order Gemellales, the order Neisseriales, the order Saprospirales, the order Actinomycetales, the order Streptophyta, the order Rhizobiales, the order Rhodospirillales, the order Xanthomonadales, the order Myxococcales, the order Campylobacterales, the order Desulfovibrionales, the order Solirubrobacterales, the order Opitutales, the order RB41, the order Rickettsiales, the order Pirellulales, the order Synergistales, the order Planctomycetales, the order Acidobacteriales, the order Solibacterales, the order Gaiellales, the order Gemmatales, the order Acidimicrobiales, the order WD2101, the order Chthoniobacterales, the order Thermoanaerobacterales, the order Pedosphaerales, the order Phycisphaerales, the order Sediment-1, the order Chlorophyta, the order iii1-15, the order Synechococcales, the order Roseiflexales, the order JG30-KF-AS9, the order Ellin329, the order Anaerolineales, the order Ellin5290, the order SC-I-84, the order Cryptophyta, the order MBNT15, the order envOPS12, the order B07_WMSP1, the order UA01, and the order Thermotogales.

12. The method of claim 8, wherein process (c) comprises comparing an increase or decrease in content of extracellular vesicles derived from one or more bacteria selected from the group consisting of the family Exiguobacteraceae, the family Enterococcaceae, the family Turicibacteraceae, the family Mogibacteriaceae, the family Moraxellaceae, the family Porphyromonadaceae, the family Burkholderiaceae, the family Actinomycetaceae, the family Coriobacteriaceae, the family Methylobacteriaceae, the family Streptococcaceae, the family Pseudomonadaceae, the family Pasteurellaceae, the family Veillonellaceae, the family Peptostreptococcaceae, the family Enterobacteriaceae, the family Verrucomicrobiaceae, the family Lachnospiraceae, the family Leuconostocaceae, the family Bradyrhizobiaceae, the family Rikenellaceae, the family Tissierellaceae, the family Bacteroidaceae, the family Chitinophagaceae, the family Corynebacteriaceae, the family Xanthomonadaceae, the family Rhizobiaceae, the family Propionibacteriaceae, the family Desulfobacteraceae, the family Barnesiellaceae, the family Comamonadaceae, the family mitochondria, the family Hyphomicrobiaceae, the family Alteromonadaceae, the family Sinobacteraceae, the family Pirellulaceae, the family Dethiosulfovibrionaceae, the family Acidobacteriaceae, the family Planctomycetaceae, the family Isosphaeraceae, the family Gaiellaceae, the family Koribacteraceae, the family Helicobacteraceae, the family Chthoniobacteraceae, the family Gemmataceae, the family C111, the family Solibacteraceae, the family Pelagibacteraceae, the family PRR-10, the family Ellin515, the family Thermoanaerobacteraceae, the family Methanoregulaceae, the family Synechococcaceae, the family Desulfomicrobiaceae, the family Kouleothrixaceae, the family OCS155, the family Alicyclobacillaceae, the family Myxococcaceae, the family EB1017, the family Anaerolinaceae, and the family Desulfohalobiaceae.

13. The method of claim 8, wherein process (c) comprises comparing an increase or decrease in content of extracellular vesicles derived from one or more bacteria selected from the group consisting of the genus Collinsella, the genus Adlercreutzia, the genus SMB53, the genus Proteus, the genus Exiguobacterium, the genus Enterococcus, the genus Acinetobacter, the genus Turicibacter, the genus Klebsiella, the genus Lautropia, the genus Akkermansia, the genus Parabacteroides, the genus Rhizobium, the genus Actinomyces, the genus Lactococcus, the genus Blautia, the genus Veillonella, the genus Pseudomonas, the genus Rothia, the genus Dorea, the genus Streptococcus, the genus Haemophilus, the genus Enhydrobacter, the genus Rhodococcus, the genus Coprococcus, the genus Oscillospira, the genus Ruminococcus, the genus Bacteroides, the genus Corynebacterium, the genus Weissella, the genus Propionibacterium, the genus Lysinibacillus, the genus Stenotrophomonas, the genus Arthrobacter, the genus Comamonas, the genus Marinobacter, the genus Clostridium, the genus Planctomyces, the genus Luteolibacter, the genus Delftia, the genus Agrobacterium, the genus Rhodoplanes, the genus DA101, the genus Gemmata, the genus Coprobacillus, the genus Arcobacter, the genus Helicobacter, the genus Candidatus Solibacter, the genus Methanosarcina, the genus Thermacetogenium, the genus Synechococcus, the genus Desulfomicrobium, the genus Chthoniobacter, the genus Aminobacterium, the genus Gallicola, the genus Anaeromyxobacter, the genus Muricauda, and the genus Candidatus Koribacter.

14. The method of claim 8, wherein the subject sample is urine.


SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc